(19) United States
(12) Patent Application Publication
(10) Pub. No.: US 2011/0201631 A1
(43) Pub. Date: Aug. 18, 2011

(54) PHARMACEUTICAL FORMULATIONS COMPRISING PEMETREXED

(75) Inventors: Chandrasekhar Kocherlakota, Secunderabad (IN); Raghuveera Hathibelagal Goruva, Kurnool (IN); Krishnam Raju Kovoru, East Godavari (IN); Nagaraju Banda, Hyderabad (IN)

(73) Assignees: DR. REDDY'S LABORATORIES LTD., Hyderabad (IN); DR. REDDY'S LABORATORIES, INC., Bridgewater, NJ (US)

(21) Appl. No.: 13/044,095

(22) Filed: Mar. 9, 2011

Related U.S. Application Data
(63) Continuation of application No. PCT/US2009/056211, filed on Sep. 8, 2009.
(60) Provisional application No. 61/139,189, filed on Dec. 19, 2008.

(30) Foreign Application Priority Data
Sep. 11, 2008 (IN) 2207/CHE/2008

(51) Int. Cl. A61K 31/519; C07D 487/04; A61P 35/00

(52) U.S. Cl. 514/265.1; 544/280

(57) ABSTRACT

Pharmaceutical formulations comprising amorphous pemetrexed or its salts, and processes to prepare the formulations.

### INTRODUCTION

Aspects of the present invention relate to pharmaceutical formulations comprising pemetrexed or its pharmaceutically acceptable salts or solvates or hydrates, in the form of ready-to-use solutions or in lyophilized forms. Aspects of the invention include pharmaceutical formulations comprising pemetrexed or its salts, solvates, or hydrates, in the form of ready-to-use solutions or in lyophilized forms, and the preparation thereof, where X-ray diffraction patterns do not show any diffraction peaks that allow calculation of ‘d’ spacings of about 7.78±0.04 Å. Further aspects of the invention include pharmaceutical formulations comprising amorphous pemetrexed or its salts or its hydrates. Various aspects of the invention also relate to stable pharmaceutical formulations comprising pemetrexed or its salts or hydrates, processes for preparing such formulations and methods of using them for treating various cancers in mammals.

Cancers, including estrogen dependent cancers, are generally thought to result from a multi-step process, in which a series of somatic mutations, and/or chromosomal changes occur. Each step results in a greater deviation from normal cellular behavior, until cells lose the normal ability to regulate their own growth and therefore proliferate. The altered cells first proliferate into a precancerous neoplasm, which progresses in stages toward metastatic cancer. This process is known as tumor progression.

Pyrrolo[2,3-d]pyrimidine based antifolates have been used for a number of years as chemotherapeutic agents in the treatment of cancer. Pemetrexed is a 5-substituted pyrrolo[2,3-d]pyrimidine disodium salt.

Pemetrexed disodium heptahydrate has a chemical name L-Glutamic acid, N-[4-[2-(2-amino-4,7-dihydro-4-oxo-1H-pyrrolo[2,3-c]pyrimidin-5-yl)ethyl]benzoyl]-, disodium salt, heptahydrate. The compound can be represented by structural formula I.

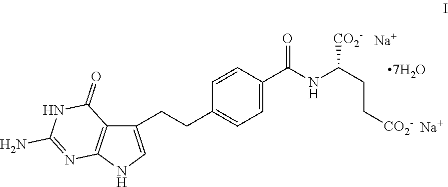

Pemetrexed is an anti-folate anti-neoplastic agent that exerts its action by disrupting folate-dependent metabolic processes essential for cell replication. It is believed to work by inhibiting three enzymes that are required in purine and pyrimidine biosynthesis: thymidylate synthase (TS); dihydrofolate reductase (DHFR); and glycinamide ribonucleotide formyl transferase (GARFT).

Pemetrexed is available in products from Eli Lilly and Company sold as ALIMTA® sterile lyophilized powders for intravenous infusion, available in single-dose vials containing 100 or 500 mg of pemetrexed equivalent. ALIMTA is indicated for locally advanced or metastatic nonsquamous non-small cell lung cancer (by initial treatment in combination with cisplatin, maintenance treatment of patients whose disease has not progressed after four cycles of platinum based first-line chemotherapy) and mesothelioma in combination with cisplatin. U.S. Pat. No. 7,138,521 describes a stable crystalline heptahydrate form of pemetrexed having a characteristic X-ray diffraction pattern, which comprises a peak corresponding to a ‘d’ spacing of 7.78±0.04 Å when obtained at 22±2° C. and at ambient relative humidity. The patent states that pemetrexed can exist in the form of a heptahydrate which is much more stable than the previously known 2.5 hydrate and shows that the primary advantage of the heptahydrate crystalline form over the 2.5 hydrate crystal form is its stability and also with respect to formation of related substances. It also shows that when the heptahydrate is subjected to elevated temperatures, low humidity, and/or vacuum, it converts to the 2.5 hydrate crystal form by loss of water.

The above patent shows that problems may arise because of conversions between different polymorphic forms of pemetrexed when exposed to elevated temperatures, low humidity, etc. Formulation processes may involve a variety of the above mentioned adverse conditions, resulting in a possibility that the stability of the final product may be affected.

The manufacturer of ALIMTA reports 24 hour stability for reconstituted solutions or infusions. It is important to obtain information on the extended stability of the drug in order to store vials and infusions in a centralized reconstituted depot for cytotoxic infusions. The extended stability of prepared infusions can avoid waste in cases where treatment is deferred after infusion preparation.

Formulating pemetrexed has not proven to be an easy task, due to its stability issues. It is known that amorphous forms of active ingredients can be relatively more unstable, when compared to the crystalline form. Thus, stabilizing amorphous pemetrexed or its salts in formulations is considered difficult.

There remains a need for preparing pemetrexed formulations with improved stability.

### SUMMARY

Aspects of the present invention relate to pharmaceutical formulations comprising pemetrexed or its pharmaceutically acceptable salts or solvates or hydrates, in the form of ready-to-use solutions or lyophilized forms. Aspects of the invention include pharmaceutical formulations comprising pemetrexed or salts or solvates or hydrates, in the form of ready-to-use solutions or lyophilized forms and preparations thereof, whose X-ray diffraction patterns do not show any diffraction peaks that allow calculation of ‘d’ spacings about 7.78±0.04 Å. Aspects of the invention relate to stable pharmaceutical formulations comprising pemetrexed or its salts or hydrates, processes for preparing such formulations and methods of using them for treating various cancers in mammals.

In embodiments, the invention includes pharmaceutical formulations comprising amorphous pemetrexed or its salts.

In embodiments, the invention includes stable pharmaceutical formulations comprising amorphous pemetrexed or its salts.

In embodiments the invention relates to stable pharmaceutical formulations of pemetrexed or its salts, wherein at least about 50% by weight of the pemetrexed or salt is in amorphous form.

In embodiments, the invention relates to stable pharmaceutical formulations of pemetrexed or its salts wherein pemetrexed or its salt is substantially in amorphous form.

In embodiments, the invention includes stable pharmaceutical formulations of a crystalline form of pemetrexed disodium having an X-ray diffraction pattern comprising peaks, expressed in terms of 2-theta angles, at about 5.8, 12.4, 18.3, 18.6, 19.6, 20.4, 24.5, 24.9, 25.8, 28.9, 29.2, 29.6, and 32.8, ±0.2 degrees.

In embodiments, the invention includes stable pharmaceutical formulations of a crystalline form of pemetrexed disodium having an X-ray diffraction pattern comprising peaks, expressed in terms of 2-theta angles, at about 5.7, 12.1, 12.3, 17.7, 18.4, 20.2, 22.2, 22.5, 22.7, 24.7, 25.6, 25.8, 26.6, 28.2, 30.3, 31.3, and 31.8, ±0.2 degrees.

In embodiments, the invention includes stable pharmaceutical formulations of a crystalline form of pemetrexed disodium having an X-ray diffraction pattern comprising peaks, expressed in terms of 2-theta angles, at about 4.0, 17.3, 18, 19.5, 20.4, 21, 29, and 43.3, ±0.2 degrees.

In embodiments, the invention relates to stable solid pharmaceutical formulations of solid dispersions of pemetrexed or its salts, including hydrates thereof that include pemetrexed or its salts and a pharmaceutically acceptable carrier.

In embodiments, the invention relates to solid pharmaceutical formulations comprising pemetrexed or its salts, including hydrates thereof, wherein a moisture content of the formulation is less than about 8% by weight.

In embodiments, the invention relates to pharmaceutical formulations comprising pemetrexed or its salts, including hydrates thereof, wherein total drug-related impurities in the formulation are less than about 3% by weight of the label content of pemetrexed or its salts, or their hydrates.

In embodiments, the invention relates to pharmaceutical formulations of pemetrexed disodium, wherein the pemetrexed disodium remains in its original amorphous form during storage at 40° C. and 75% relative humidity (RH) for at least 3 months.

In embodiments, the present invention provides simple, rapid and inexpensive manufacturing processes for preparing stable ready-to-use solutions comprising pemetrexed or its salts, or their hydrates.

In embodiments, the present invention provides methods for preparing stable lyophilized formulations comprising pemetrexed or its salts, or their hydrates.

In embodiments, the present invention provides lyophilized amorphous pemetrexed pharmaceutical formulations, suitable for treating cancers.

In embodiments the invention provides sugar free or mannitol free compositions of pemetrexed or its salts, or their hydrates.

An embodiment provides a process for preparing a pharmaceutical formulation comprising pemetrexed or a salt thereof, or a hydrate thereof, and at least one pharmaceutically acceptable excipient, comprising removing solvent from a solution comprising pemetrexed or a salt thereof to produce a product having pemetrexed or a salt thereof substantially in amorphous form.

### BRIEF DESCRIPTION OF THE DRAWINGS

FIG. 1 is a graphical comparison of segments of X-ray powder diffraction (XRD) patterns for the commercially available ALIMTA pemetrexed disodium-containing product (A) and a product of Example 1 (B).

FIG. 2 shows comparative XRD patterns of: crystalline pemetrexed disodium (A); a composition prepared according to Example 4 (B); a composition prepared according to Example 4, after exposure to 30° C. and 75% relative humidity (“RH”) conditions for three months (C); a composition prepared according to Example 4, after exposure to 40° C. and 75% RH conditions for three months (D); and a composition prepared according to Example 4, but omitting pemetrexed disodium (E).

FIG. 3 shows comparative XRD patterns of: crystalline pemetrexed disodium (F); a composition prepared according to Example 5 (G); a composition prepared according to Example 5, after exposure to 30° C. and 75% RH conditions for three months (H); a composition prepared according to Example 5, after exposure to 40° C. and 75% RH conditions for three months (I); and a composition prepared according to Example 5, but omitting pemetrexed disodium (J).

### DETAILED DESCRIPTION

In aspects, the present invention relates to pharmaceutical compositions with comprising pemetrexed improved stability comprising pemetrexed, including its pharmaceutically acceptable salts or solvates, in the form of ready-to-use solutions or lyophilized forms. Aspects of the invention include pharmaceutical compositions comprising pemetrexed, including its pharmaceutically acceptable salts or solvates, in the form of ready-to-use solutions or lyophilized forms and preparations thereof for parenteral administration, having X-ray diffraction patterns that do not include any diffraction peaks which allow calculation of ‘d’ spacings about 7.78±0.04 Å. Aspects of the invention also include processes for preparing stable amorphous pemetrexed pharmaceutical formulations suitable for parenteral administration. Aspects also include processes for preparing such compositions and methods of using such compositions for treating various cancers in mammals. The terms ‘stable’ or ‘stability’ as used herein relate to both physical and chemical stability, wherein pemetrexed or its salts or hydrates can be stored for commercially significant periods, such as lat least 3 months, 6 months, 1 year, or 2 years, without significant chemical degradation or transformation of its physical form. Percent degradation may be determined by analyzing for impurities.

The term “pharmaceutically acceptable” refers to ingredients that are useful for preparing pharmaceutical compositions, and that is considered to be generally safe, non-toxic and neither biologically nor otherwise undesirable, and includes those ingredients acceptable for veterinary use as well as human pharmaceutical use.

Pemetrexed disodium used to prepare compositions can be in a crystalline form, such as a heptahydrate or a 2.5 hydrate, or in an amorphous form.

The term “substantial” as used to describe polymorphic purity of amorphous pemetrexed means at least about 90%, or at least about 95%, or at least about 99%, amorphous form.

Injectable formulations are frequently formulated as aqueous solutions, in which water is the primary excipient. Injectable formulations can be prepared in conventional forms, either as liquid solutions or suspensions, solid forms suitable for solubilization or suspension in liquid prior to injection, or as emulsions. Sterile injectable formulations can be prepared according to techniques known in the art using suitable carriers, dispersing or wetting agents, and suspending agents. The injectable formulations may be sterile injectable solutions or suspensions in a nontoxic, parenterally acceptable diluent or solvent. Among the acceptable vehicles and solvents that may be employed are water, Ringer's solution, and isotonic sodium chloride solution. In addition, sterile, fixed oils, fatty esters or polyols are conventionally employed as solvents or suspending media.

The formulations of the present invention are particularly suited for use in parenteral administration, but it will be understood that the solutions may have alternative uses. For example, they may be used as intermediates in the preparation of other pharmaceutical dosage forms. Similarly, they may have other routes of administration including intranasal or inhalation. Injectable formulations may take any route including intramuscular, intravenous or subcutaneous.

Also provided herein are processes for the preparation of injectable pharmaceutical formulations. Certain processes include lyophilizing or freeze-drying an aqueous solution, such as an alkaline or acidic aqueous solution, which comprises pemetrexed or its salts or hydrates and pharmaceutically acceptable excipients.

A single compound may give rise to a variety of solid forms having distinct physical properties. Different solid forms of the same drug may exhibit different properties.

X-ray powder diffraction (“XRD”) is one of the primary techniques used by solid state chemists to examine the physico-chemical nature of unknown solids. Each crystalline solid has its unique characteristic X-ray powder diffraction pattern which may be used as a “fingerprint” for its identification. Once the material has been identified, X-ray crystallography may be used to determine its structure, i.e., how the atoms pack together in a crystalline state and what the interatomic distances and angles are.

The XRD technique uses a powdered sample in a holder, the sample is subjected to X-ray radiation of a fixed wavelength, and the intensity of the reflected radiation is recorded at various angles using a goniometer. The reflection angles are used to calculate the inter-atomic spacing (‘d’ values in Angstrom units, 10⁻⁸ cm). The peak intensities (I) are measured to discriminate (using I-ratios) the various ‘d’ spacings.

X-ray powder diffraction spectroscopic results reported herein were obtained using copper K alpha radiation. The specimens for analysis were powdered and packed in the sample holder, using back loading. The specimens were exposed to the room environment with ambient temperature and humidity.

Sample preparation was minimal, to prevent polymorphic form changes. Sample particles were lightly packed into the sample holder to insure that they formed a smooth surface and did not clump together.

A pemetrexed lyophilized formulation prepared according to the invention and a commercially available lyophilized formulation of pemetrexed can be subjected to X-ray powder diffraction analysis with optimized parameters as shown in Table 1, below, wherein the samples are slowly scanned between 10.7 and 12.4 degrees 28 values. A d-spacing about 7.78 Å would be indicated by a peak at about 11.4-11.5 degrees 2θ.

Pemetrexed lyophilized formulations according to the invention exhibit an amorphous nature after lyophilization which can be ascertained by their XRD patterns, whereas current commercial formulations of pemetrexed have traces of crystalline material as ascertained by their XRD patterns, which indicates that the present formulations are more stable with respect to the conversions of polymorphs, even if exposed to adverse conditions after being subjected to a lyophilization process.

It has been discovered that a simple, isotonic saline solution of pemetrexed is not pharmaceutically acceptable for commercial purposes due to degradation of the solution to form unacceptable related substances.

Impurities or related substances or degradants in any formulation are undesirable, and, in extreme cases, might even be harmful to a patient. Further these undesired impurities may reduce the availability of the API to elicit the pharmacological effect and often affect the stability of the dosage form. Hence the levels of these impurities should be maintained at low levels in the formulation throughout the shelf life of the product. Commercially acceptable shelf life can be at least 3 months, 6 months, 1 year, 2 years, etc.

Pemetrexed appears to be susceptible to oxidation and the presence of moisture; hence its formulations should be processed and maintained at minimum levels of oxygen. The headspace of a vial should contain less than about 8% (eight percent) v/v oxygen, or in the range of about 2% to about 5% v/v oxygen, or in the range of about 3% to about 5% v/v oxygen. The headspace of the vial can be adjusted to minimize the formulation contact with oxygen. It is generally desired that the headspace is not more than about one-third of the total volume of the container, with the contents occupying at least about two-thirds of the total volume of the container. For example, 5 mL of product may be contained in a 7.5 ml vial. To avoid oxidation, antioxidants can be included. If a greater headspace ratio is desired, then the concentration of an antioxidant may be adjusted as necessary.

Common antioxidants that can be used in the formulation include, but are not limited to, monothioglycerol, L-cysteine, thioglycolic acid, sodium metabisulfite, ascorbic acid, sodium EDTA, monoethanolamine gentisate, sodium formaldehyde sulfoxylate, sodium bisulfite, and the like.

The following pemetrexed-related degradants or impurities have been observed:

1) “Pemetrexed Impurity 7,” having a chemical name 4-[2-(2-amino-4-oxo-4,7-dihydro-3H-pyrrolo[2,3-d]pyrimidin-5-yl)-ethyl]-benzoic acid, and represented by structural formula II.

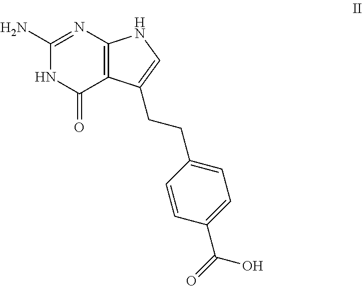

2) “Pemetrexed Impurity 8,” having a chemical name N-4-[2(2-amino-4,7-dihydro-4-oxo-1H-pyrrolo[2,3-d]pyrimidin-5-yl)ethyl] benzoyl-L-glutamic acid dimethyl ester p-toluene sulphonic acid salt, and represented by structural formula III.

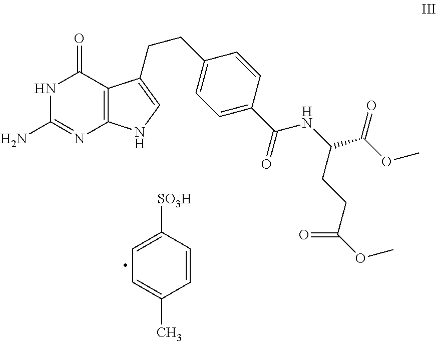

3) A chiral impurity of pemetrexed disodium, represented by structural formula IV.

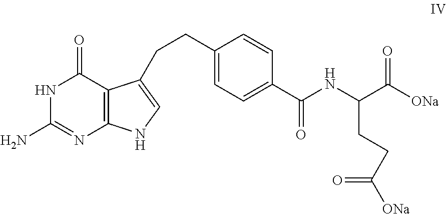

4) “Impurity A,” represented by structural formula V.

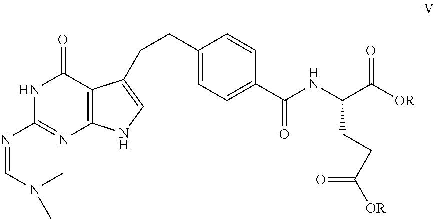

5) “Impurity B,” represented by structural formula VI.

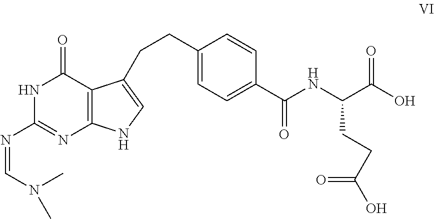

In embodiments, the invention includes pharmaceutical formulations comprising amorphous pemetrexed or its salts, or hydrates thereof.

In embodiments, the invention includes stable pharmaceutical formulations comprising amorphous pemetrexed or its salts, or hydrates thereof.

In embodiments, the invention relates to stable pharmaceutical formulations of pemetrexed or its salts, wherein at least about 50% by weight of the pemetrexed or its salts is in amorphous form.

In embodiments, the invention relates to stable pharmaceutical formulations of pemetrexed or its salts, wherein at least about 75% by weight of the pemetrexed or its salts is in amorphous form.

In embodiments, the invention relates to stable pharmaceutical formulations of pemetrexed or its salts, wherein at least about 90% by weight of the pemetrexed or its salts is in amorphous form.

In embodiments, the invention relates to stable pharmaceutical formulations of pemetrexed or its salts, wherein at least about 95% by weight of the pemetrexed or its salts is in amorphous form.

In embodiments, the invention relates to stable pharmaceutical formulations of solid dispersions of pemetrexed or its salts, which comprise pemetrexed or its salts and a pharmaceutically acceptable carrier.

Aspects of the invention relate to processes for making amorphous pemetrexed or its salts, in the form of a free solid or as a solid dispersion with at least one pharmaceutically acceptable carrier, an embodiment comprising:

i) providing a solution of pemetrexed or its salts, alone or in combination with a pharmaceutically acceptable carrier, in a solvent; and

ii) removing the solvent.

Suitable pharmaceutically acceptable carriers include, but are not limited to: cellulose derivatives, such as hydroxypropyl methylcelluloses and hydroxypropyl celluloses; polyvinylpyrrolidones; sugars such as sucrose, mannose, glucose, and the like; sugar alcohols, such as mannitol, sorbitol, and the like; gums; cyclodextrins; gelatins; hypromellose phthalates; polyhydric alcohols; polyethylene glycols; polyethylene oxides; polyoxyethylene derivatives; polyvinyl alcohols; propylene glycol derivatives; and any mixtures thereof.

Suitable solvents that can be used for preparing amorphous pemetrexed or its salts include water and any organic solvents from the various classes of solvents, such as, for example, alcohols, ketones, esters, ethers, halogenated hydrocarbons, aromatic hydrocarbons, nitriles, aprotic polar solvents, acidic solvents, and mixtures of any two or more thereof. Useful alcohols include, for example, methanol, ethanol, denatured spirits, n-propanol, isopropanol, n-butanol, isobutanol, t-butanol, and the like. Useful ketones include acetone, propanone, 2-butanone, and the like. Useful halogenated hydrocarbons include, for example, dichloromethane, 1,2-dichloroethane, chloroform, carbon tetrachloride, chlorobenzene, and the like. Useful esters include, for example, ethyl acetate, n-propyl acetate, isopropyl acetate, n-butyl acetate, t-butyl acetate, and the like. Useful ethers include, for example, dimethyl ether, diethyl ether, methyl t-butyl ether, ethyl methyl ether, diisopropyl ether, tetrahydrofuran, dioxane, and the like. Useful aromatic hydrocarbons include, for example, toluene, xylene, and the like. Useful nitriles include acetonitrile, propionitrile, and the like. Useful aprotic polar solvents include N,N-dimethylformide (DMF), dimethylsulfoxide (DMSO), N,N-dimethylacetamide (DMA), and the like. Useful acidic solvents include formic acid, acetic acid, and the like. This listing is not intended to be exhaustive, and combinations of solvents that are useful can include more than one member of a class, and/or can be from different classes.

These and other classes of solvents known to a person skilled in the art are all contemplated without limitation. The organic solvents acceptable for the practice of the process described herein will provide sufficient solubility for the active substance, and do not cause any undesirable chemical reactions with the pemetrexed or its salt, such as degradation, under the conditions of processing.

Amorphous pemetrexed or its salts may be obtained by drying a solution of pemetrexed or its salts in a solvent. Conventional processes, such as freeze drying or lyophilization, spray drying, flash drying, distillation using a rotational evaporator device such as a Buchi Rotavapor, agitated thin film drying (ATFD), and the like may be used for recovering amorphous pemetrexed or its salts from a solution of pemetrexed or its salts. These techniques are applicable to both aqueous and nonaqueous solutions of pemetrexed disodium, and mixtures of pemetrexed disodium with a pharmaceutically acceptable carrier.

The technique known as lyophilization is often employed for injectable pharmaceuticals, which exhibit poor stability in aqueous solutions. Lyophilization processing is suitable for injectables because it can be conducted in sterile conditions, which is primary requirement for parenteral dosage forms. During the lyophilization process, a complex structure could become damaged. Such damage can be prevented by the use of cryoprotectants. Cryoprotectants for use in the present invention include all of the pharmaceutically acceptable carriers, which may be used in the invention.

Lyophilization or freeze drying is a process in which water is removed from a product after it is frozen and placed under a vacuum, allowing the ice to change directly from a solid to a vapor state, without passing through a liquid state. The process consists of three separate, unique, and interdependent processes: a freezing phase, a primary drying phase (sublimation), and a secondary drying phase (desorption). These processes may be optimized to enhance the product stability as well as decrease the manufacturing costs.

### Freezing Phase:

The primary function of the freezing phase is to ensure that the entire container with the complex solution is completely frozen prior to proceeding to the primary dry phase. Additionally, it is preferable that these materials freeze in a uniform manner. While there are different ways that this can be accomplished, one option is to chill the containers after they are loaded onto the lyophilizer shelves and hold for 30-60 minutes prior to initiation of the freezing cycle. It is generally not practical to equilibrate the shelves to a freezing temperature, because of frost accumulation during the filling and loading of the containers.

### Primary Drying Phase:

Once a material is brought to the desired frozen state, primary drying via sublimation can proceed. The primary dry phase involves the removal of bulk water at a product temperature below the ice transition temperature under a vacuum with pressures typically between 50-150 milliTorr (6.7-20 Pa). This phase important for stabilizing the active. It is helpful to identify the glass transition temperature (Tg′) for the formulation. The Tg′ is the temperature at which there is a reversible change of state between a viscous liquid and a rigid, amorphous glassy state. One can measure the Tg′ of candidate formulations using a differential scanning calorimeter (DSC), in particular with modulated DSC. Generally, the collapse temperature is observed to be about 2-5° C. greater than the Tg′. Hence, the shelf temperature is set such that the target product temperature is maintained near or below the Tg′ of the formulation throughout the removal of solvent during the primary dry phase.

As the solvent is progressively removed from the formulated containers, the product temperature will approach and reach the shelf temperature since it is no longer cooled by water sublimation. To optimize the duration of the primary drying phase, the removal of solvent vapor can be tracked using a moisture detector, or by monitoring the decrease in pressure difference between a capacitance manometer and a thermocouple pressure gauge or by a pressure drop measurement. The optimization of the primary dry cycle involves the removal of solvent as quickly as possible without causing cake collapse and subsequent product instability.

### Secondary (Terminal) Dry Phase:

The secondary drying phase is the final segment of the lyophilization cycle where residual moisture is removed from the formulation interstitial matrix by desorption with elevated temperatures and/or reduced pressures. The final moisture content of a lyophilized formulation, which can be measured using Karl Fisher or other methods, is important to determine, because if the cake contains too much residual moisture, the stability of the active can be compromised. Hence, it is imperative that one achieves a moisture level as low as possible.

To accomplish a low residual moisture, the shelf temperature is typically elevated to accelerate desorption of water molecules. The duration of the secondary dry phase is usually short. When microstructure collapse occurs, the residual moisture is generally significantly greater than desired. One alternative is to purge the sample chamber of the lyophilizer with alternating cycles of an inert gas, such as nitrogen, to facilitate displacement of bound water. However, another solution is to properly formulate the drug product and run an optimal lyophilization cycle.

The advantages of lyophilization include: ease of processing a liquid, which simplifies aseptic handling; enhanced stability of a dry powder; removal of water without excessive heating of the product; enhanced product stability in a dry state; and rapid and easy dissolution of reconstituted product. Also, the product is dried without elevated temperatures, thereby eliminating adverse thermal effects, and then stored in the dry state in which there are relatively few stability problems.

Additionally freeze dried products are often more soluble and/or more rapidly dissolved, dispersions are stabilized, and products subject to degradation by oxidation or hydrolysis are protected.

An example of a lyophilization process includes the following steps:

1) Preparing a complex solution as discussed above.

2) Sterilizing the bulk solution by passing filter.

3) Filling into individual sterile containers and loosely stoppering the containers under aseptic conditions.

4) Transporting the partially stoppered containers to the lyophilizer and loading into the chamber under aseptic conditions.

5) Conducting a lyophilization cycle, comprising a freezing phase, primary drying, and secondary drying.

6) Applying a vacuum to the chamber and heating the shelves in order to evaporate the water from the frozen state.

7) Final stoppering of the containers, such as by hydraulic or screw rod stoppering mechanisms installed in the lyophilizers.

Pharmaceuticals to be freeze dried are usually in solution having a 0.01 to 40% concentration of total solids. Usually the improvement in stability of the lyophilizate, compared to the solution, is due to the absence of water in the pharmaceutical composition.

The active constituent of many pharmaceutical products, though, is present in such small quantities that if freeze dried alone, it may not give a composition of suitable bulk and in some cases its presence would be hard to detect visually. Therefore excipients are often added to increase the amount of solids present. In most applications it is desirable for the dried product cake to occupy essentially the same volume as that of the original solution. To achieve this, the total solids content of the original solution is usually made to be about 10 to 25%.

Among the substances found useful for this purpose, often in combination, are sodium or potassium phosphates, citric acid, tartaric acid, gelatin, lactose and other carbohydrates such as dextrose, mannitol and dextran and, on occasion, preservatives. Various excipients contribute appearance characteristics to the cake, such as making it dull and spongy or sparkling and crystalline, firm or friable, expanded or shrunken, and uniform or striated. Therefore formulation of a composition to be freeze dried must include consideration not only of the nature and stability characteristics required during the liquid state, both freshly prepared and when reconstituted before use, but the characteristics desired in the final lyophilized cake.

Additionally, for products to be reconstituted for parenteral usage, consideration should also be given to the pharmacological effects of excipients chosen. In some instances there may even be chemical interaction between an active ingredient and one or more of the excipients during processing. This could, of course, result in reduced potency of the finished product. For all the above reasons, it becomes apparent that selection of a suitable excipient or excipients for a pharmaceutical product containing pemetrexed or its salts is important.

The formulation, size and shape of the vial, number of vials and type of lyophilizer will control the time required to complete primary drying, which may vary from few hours up to several days. Upon completion of primary drying the shelf temperature is raised to the desired setting to perform secondary drying.

In an embodiment, the invention includes the parameters which are of concern for lyophilized composition, wherein the resulting cake (lyophilized product) is evaluated visually on its physical appearance using as desired criteria: original shape, no shrinkage or meltback, good coloration, homogeneity, firmness and crystallinity. After the lyophilization process is completed, the material remaining in the vial is observed for color appearance, texture, friability, and shrinkage from the original volume. Also each formulation is tested for its moisture loss on drying and its dissolution characteristics. dose uniformity, sterility testing, etc.

The residual moisture levels in the lyophilized composition impact the storage stability of the lyophilized composition for a desired temperature and duration. Desirably, the amount of residual moisture in the lyophilized composition should be less than about 8% w/w, or less than about 6% w/w.

In an embodiment, the ratio of cake height to vial height is in the range of about 20 to 45%.

Reconstitution of the lyophilized composition (which can be stored for an extended period of time at typical storage temperatures), typically just before administration to the patient, utilizes an appropriate liquid medium to produce a solution, suspension, dispersion, or emulsion. A reconstitution medium may include sterile water, water for injection, a pH buffered solution, or 5% dextrose solution (D5W). The reconstitution is usually performed at room temperature, however other temperatures may also be considered. The reconstituted lyophilized composition should pass the current United States Pharmacopeia (USP) Test 788 particulate matter specifications.

The USP particulate matter test defines the amount of foreign particulate matter, as observed by optical microscopy. According to Test 788, the limit in each product container for foreign particulate matter having sizes greater than or equal to 10 μm is 3000, and for particles having sizes greater than or equal to 25 μm is 300.

The amorphous pemetrexed or its salts and its formulations are further characterized for physical parameters such as particle size distribution, bulk density, tapped density, moisture content, etc.

An important physicochemical characteristic of particulate compositions is the density properties. Bulk density is described as untapped or tapped. Untapped bulk density of a substance is the undisturbed packing density of that substance and tapped bulk density relates to the packing density after tapping a bed of substance until no change in the packing density is seen. Bulk density and tapped density can be determined using a compendial bulk density apparatus, a suitable method being given in United States Pharmacopeia 29, United States Pharmacopeial Convention, Inc., Rockville, Md., 2005, at pages 2638-2639.

The injectable pharmaceutical formulations may optionally include one or more pharmaceutically acceptable excipients. These pharmaceutically acceptable excipients may include one or more of: diluents or bulking agents such as dextrose, sucrose, mannose, mannitol and the like; antibacterial preservatives, including one or more of phenylmercuric nitrate, thiomersal, benzalkonium chloride, benzethonium chloride, phenol, cresol and chlorobutanol; chelating agents such as ethylenediamine tetraacetic acid (EDTA); buffers including one or more of acetate, citrate, tartarate, phosphate, benzoate, and bicarbonate buffers, and amino acids such as glutamic acid and histidine; tonicity contributors including one or more of sodium chloride, potassium chloride, dextrose, mannitol, sorbitol, and lactose; and alkaline substances including one or more of salts of alkali and alkaline earth metals such as sodium hydroxide, potassium hydroxide, sodium carbonate, sodium bicarbonate, and sodium phosphates, as well as organic amines such as meglumine and tromethamine.

The addition of a sugar or sugar alcohol can improve the stability of pemetrexed formulations. In various embodiments, a sugar or sugar alcohol is present in concentrations from about 10 mg/mL to about 80 mg/mL.

In embodiments, the invention provides pharmaceutically stable lyophilized formulations of pemetrexed comprising: a) pemetrexed or its salts, or hydrates thereof; and b) at least one pharmaceutically acceptable carrier.

In embodiments, the invention provides methods of formulating pharmaceutically stable solutions of pemetrexed, comprising admixing: a) pemetrexed or a pharmaceutically acceptable salt thereof; and b) a pharmaceutically acceptable carrier.

In embodiments, the invention provides stable lyophilized or ready to use solutions of pemetrexed comprising sugar and sugar alcohol free compositions of pemetrexed or a pharmaceutically acceptable salt thereof, such as mannitol free compositions of pemetrexed or a pharmaceutically acceptable salt thereof.

A stable pemetrexed formulation of this invention can be in the form of a ready-to-use dosage form, or can be in the form of a lyophilized preparation, which can be reconstituted by mixing with a diluent before administration.

In embodiments, the invention includes stable pharmaceutical formulations of a crystalline form of pemetrexed disodium having an X-ray diffraction pattern comprising peaks, expressed in terms of 2-theta angles, at about 5.8, 12.4, 18.3, 18.6, 19.6, 20.4, 24.5, 24.9, 25.8, 28.9, 29.2, 29.6, and 32.8, ±0.2 degrees.

In embodiments, the invention includes stable pharmaceutical formulations of crystalline Form B of pemetrexed disodium having an X-ray diffraction pattern comprising peaks, expressed in terms of 2-theta angles, at about 5.7, 12.1, 12.3, 17.7, 18.4, 20.2, 22.2, 22.5, 22.7, 24.7, 25.6, 25.8, 26.6, 28.2, 30.3, 31.3, and 31.8, ±0.2 degrees.

In embodiments, the invention includes stable pharmaceutical formulations of crystalline Form A of pemetrexed disodium having an X-ray diffraction pattern comprising peaks, expressed in terms of 2-theta angles, at about 4, 17.3, 18, 19.5, 20.4, 21, 29, and 43.3, ±0.2 degrees.

In embodiments, the invention relates to stable pharmaceutical formulations comprising pemetrexed or its salts or hydrates, wherein a moisture content of the formulation is less than about 8% w/w.

The formulations of the present invention are generally prepared according to conventional techniques and pH of the final formulation is adjusted to a desired value by adding an acid or base, as appropriate.

In embodiments, a solution of pemetrexed disodium in water, prior to drying, has pH in the range of 6 to about 8.

In embodiments, pH of a 2.5% w/v solution of the formulations is in the range of about 5 to 9.

In embodiments, the invention relates to freeze drying processes for removing solvent from solutions of pemetrexed or its salts.

Freeze drying can be conducted at temperatures about −40° C. to about 40° C., under vacuum in the range of about 5 to about 350 milliTorr (0.7-47 Pa). The freeze drying typically is conducted for about 10 to about 60 hours, or 15 to about 50 hours, although shorter or longer times can be used.

In embodiments, the invention provides methods of filling containers that contain a solution or lyophilized powder of pemetrexed or salts or hydrates, comprising: a) providing one or more open containers; b) filing the containers with a solution or lyophilized powder of pemetrexed, optionally in an aseptic environment; c) sealing the filled containers; and d) sterilizing the sealed, filled containers.

Vials are small glass containers that are sealed with a suitable stopper and seal, and other suitable primary containers may be used, for example, but not limited to, pre-filled syringes. Vials can be sealed containers of medication that are used one time only, and include breakable and non-breakable closed glass containers, breakable plastic containers, miniature screw-top jars, and any other type of container, typically of a size capable of holding only one unit dose of pemetrexed.

The invention includes use of packaging materials such as containers and closures of high-density polyethylene (HDPE), low-density polyethylene (LDPE) and or polypropylene and/or glass, glassine foil, polyvinyl chloride, polyvinylidene dichloride, etc.

Any pharmaceutically acceptable stopper may be used to seal the vial containing the formulation. Some of the stopper materials include silicone rubber, Teflon coated stoppers, slotted bromobutyl rubber, etc.

Mention of pemetrexed is intended to include any of the alternative forms in which the pemetrexed can be administered, such as salts, esters, hydrates, solvates, crystalline or amorphous polymorphs, racemic mixtures, enantiomeric isomers, etc.

The invention includes analytical methods for analysis of pemetrexed-related substances, using high performance liquid chromatography (HPLC), wherein a specific method comprises:

Mobile phase A: Dissolve 1.36 g of potassium di hydrogen phosphate in 1000 ml of Milli-Q water, adjust pH of the buffer to 3.0 with ortho phosphoric acid, and then filter and degass.

Mobile phase B: Acetonitrile.

Diluent: Milli-Q water

Chromatographic system:

230 nm UV detector.

Column: BDS HYPER SIL 150×4.6 mm, 5 μm.

Column temperature: 30° C.

Flow rate: 1.0 mL per minute.

Injection volume: 10 μL.

Run time: 45 minutes.

Gradient Program:

| | % Mobile | % Mobile |
|---|---|---|
| Time | Phase A | Phase B |
| 0 | 95 | 5 |
| 20 | 80 | 20 |
| 30 | 50 | 50 |
| 35 | 80 | 20 |
| 40 | 95 | 5 |
| 45 | 95 | 5 |

Preparation of Test Sample: the Contents of Two Product Vials are reconstituted with 40 mL of diluent and mixed to dissolve completely. Solutions from the two vials are combined, a 2.0 mL aliquot is diluted to 50 mL with diluent, then a 10 μL portion is injected into the chromatograph.

For pemetrexed impurity 7, a relative retention time (RRT, where pemetrexed=1) is about 1.33.

The following examples further describe certain specific aspects and embodiments of the invention and demonstrate the practice and advantages thereof. It is to be understood that the examples are provided only for purposes of illustration and are not intended to limit the scope of the invention in any manner.

### Example 1

### Pemetrexed Formulation

| Ingredient | mg/Vial |
|---|---|
| Pemetrexed | 500 |
| Mannitol | 500 |
| NaOH and/or HCl | q.s. |
| Water for Injection‡ | q.s. |

‡Evaporates during lyophilization.

Manufacturing Process:

1. Mannitol is dissolved in water.

2. Pemetrexed is dissolved in the mannitol solution with stirring.

3. The pH is adjusted to a desired value (e.g., isotonic with blood) by adding sodium hydroxide or hydrochloric acid solution.

4. The volume is made up to a desired quantity with water and mixed well.

5. The step 4 solution is filtered through a 0.2 μm sterile membrane filter.

6. The step 5 solution is filled into depyrogenated USP Type 1 glass vials and the vials are loosely stoppered.

7. The loosely stoppered vials are lyophilized in a freeze dryer.

8. After lyophilization, the vials are stoppered completely by hydraulic pressing and sealed with flip-off seals.

The lyophilized product can be reconstituted using sterile water for injection, prior to use.

A ready-to-use solution can be prepared using similar ingredients and a manufacturing process as above, except for the lyophilization step which is not required for the formulation of a ready-to-use solution.

When XRD patterns of the pemetrexed formulation prepared above and a marketed formulation (ALIMTA) are compared, the X-ray diffraction pattern of the Example 1 formulation does not show any diffraction peaks that allow calculation of a ‘d’ spacing of about 7.78±0.04 Å, whereas a clear and distinct elevation is observed in the XRD pattern of the ALIMTA formulation. The above results show that pemetrexed formulations of Example 1 exhibit an amorphous nature which can be ascertained by their XRD patterns, whereas ALIMTA formulations of pemetrexed contain crystalline material as ascertained by their XRD patterns.

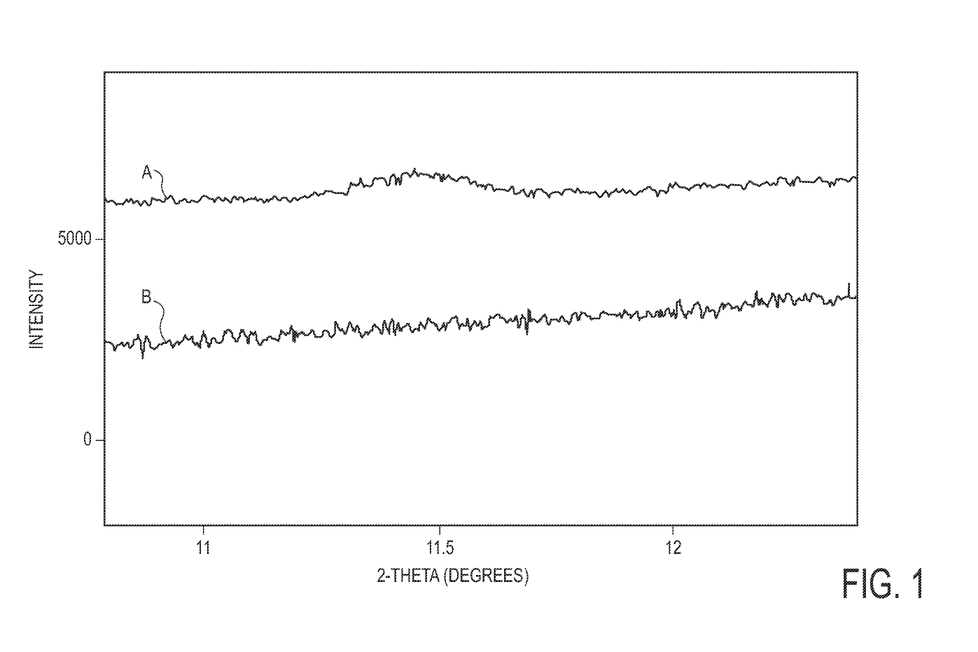

X-ray powder diffraction patterns for a lyophilized formulation prepared as above (B) and the commercially available ALIMTA product (A) are shown in FIG. 1. All of the patterns described herein have been generated using the parameters of Table 1.

TABLE 1
X-ray diffraction parameters.

| Equipment | PANalytical XPertPro |
|---|---|
| Detector | Xcelerator |
| Current, Voltage | 40 mA, 45 kV |
| Goniometer | Theta/Theta |
| Start Position [degrees 2-Theta] | 10.7561 |
| End Position [degrees 2-Theta] | 2.4961 |
| Step Size [degrees 2-Theta] | 0.004 |
| Scan Step Time [seconds] | 479.7221 |
| Scan Type | Continuous |
| PSD Mode | Scanning |
| PSD Length [degrees 2-Theta] | 2.12 |
| Offset [degrees 2-Theta] | 0 |
| Divergence Slit Type | Automatic |
| Irradiated Length [mm] | 10 |
| Specimen Length [mm] | 10 |
| Measurement Temperature [° C.] | 25 |
| Anode Material | Copper |
| K Alpha-1 [Å] | 1.5406 |
| K Alpha-2 [Å] | 1.54443 |
| K Beta [Å] | 1.39225 |
| K Alpha-2/K Alpha-1 Ratio | 0.5 |

### Example 2

### Pemetrexed Formulation

| Ingredient | mg/Vial |
|---|---|
| Pemetrexed | 500 |
| NaOH and/or HCl | q.s. |
| Water for Injection‡ | q.s. |

‡Evaporates during lyophilization.

Manufacturing Process:
1. Dissolve pemetrexed in water with stirring.
2. Adjust the pH to a desired value (isotonic with blood) by adding sodium hydroxide or hydrochloric acid solution.
3. Make up the volume to a desired quantity with water and mix well.
4. Filter the solution through a 0.2 μm sterile membrane filter to produce a sterile filtrate.
5. Fill the solution into depyrogenated USP Type 1 glass vials and loosely stopper the vials.
6. Lyophilize the loosely stoppered vials in a freeze dryer.
7. After completion of lyophilization, stopper the vials completely by hydraulic pressing and seal the vials with flip-off seals.

The lyophilized product can be reconstituted using sterile water for injection, prior to use.

A ready-to-use solution can be prepared using similar ingredients and a manufacturing process as above, except for the lyophilization step which is not required for the formulation of a ready-to-use solution.

### Example 3

### Pemetrexed 500 mg Formulation

| Ingredient | mg/Vial |
|---|---|
| Pemetrexed disodium | 698.97 |
| Mannitol | 500 |
| Sodium citrate | 10.6 |
| Glutamic acid | 0.56 |
| Water for Injection‡ | q.s. |

### Manufacturing Process:

1) About 90% of the water (temperature about 25±5° C.) is placed into a mixing vessel and stirred continuously. During stirring, the solution is purged continuously with nitrogen gas.

2) Mannitol is added to the water and dissolved with continuous stirring.

3) Sodium citrate is added to the solution and dissolved with continuous stirring. To this solution, glutamic acid is added and dissolved with continuous stirring.

4) Pemetrexed disodium is added to the solution and dissolved with continuous stirring.

5) The volume is made up to the desired volume with remaining water for injection and mixed well.

6) 20 mL of the solution from step 5) is filled into 50 mL tubular USP type I glass vials and the vials are loosely stoppered with bromobutyl stoppers.

7) The loosely stoppered vials are lyophilized in a freeze dryer, using the lyophilization cycle described below for Example 4.

### EXAMPLES 4-5

### Pemetrexed Formulation

mg/Vial

| Ingredient | Example 4 | Example 5 |
|---|---|---|
| Pemetrexed disodium | 551.425 | 110.285 |
| Mannitol | 500 | 106 |

| | |
|---|---|
| Sodium hydroxide or | q.s. |
| hydrochloric acid | |

| | | |
|---|---|---|
| Water for Injection‡ | q.s. to 20 mL | q.s. to 4 mL |

### Manufacturing Process:

1) About 90% of the water is placed into a vessel and stirred continuously, while filtered nitrogen gas is purged into the liquid.

2) Mannitol is added to the liquid with stirring to dissolve.

3) Pemetrexed disodium is added to the step 2) solution and dissolved with continuous stirring.

4) The pH of the solution is adjusted, as needed, to about 6.6 to 7.8 using 0.5 N sodium hydroxide solution or 0.5 N hydrochloric acid solution. A pH for Example 4 is observed to be 7.21 and for Example 5 is observed to be 7.19.

5) The volume is made up to 20 mL with the remaining water and mixed well.

6) For Example 4, 20 mL of the solution from step 5) is filled into 50 mL depyrogenated USP type I glass vials and the vials are loosely stoppered with slotted sterile bromobutyl rubber stoppers.

7) For Example 5, 4 mL of the solution from step 5) is filled into 10 mL USP type I glass vials and the vials are loosely stoppered with slotted sterile bromobutyl rubber stoppers.

8) The loosely stoppered vials are lyophilized in a freeze dryer.

Lyophilization cycle parameters are as follows:

| | | Shelf | Chamber | | Cumulative |
|---|---|---|---|---|---|
| | | Temp. | Pressure | Time | Time |
| Step | Hold/Ramp | (° C.) | (mT) | (minutes) | (minutes) |
| 1 | Hold | 25 | — | 2 | 2 |
| 2 | Ramp | 25 to −35 | — | 180 | 182 |
| 3 | Hold | −35 | — | 60 | 242 |
| 4 | Ramp | −35 to −10 | — | 60 | 302 |
| 5 | Hold | −10 | — | 60 | 362 |
| 6 | Ramp | −10 to −35 | — | 60 | 422 |
| 7 | Hold | −35 | — | 60 | 482 |
| 8 | Hold | −35 | — | 5 | 487 |

Primary Drying Phase

| | | | | | |
|---|---|---|---|---|---|
| 9 | Hold | −35 | 300 | 5 | 492 |
| 10 | Ramp | −35 to −15 | 300 | 120 | 612 |
| 11 | Hold | −15 | 300 | 900 | 1512 |
| 12 | Ramp | −15 to −5 | 300 | 60 | 1572 |
| 13 | Hold | −5 | 300 | 480 | 2052 |
| 14 | Ramp | −5 to 25 | 300 | 180 | 2232 |
| 15 | Hold | 25 | 300 | 180 | 2412 |

Secondary Drying Phase

| | | | | | |
|---|---|---|---|---|---|
| 16 | Hold | 35*** | 10 | 420 | 2832 |

*300 milliTorr = 40 Pa.
**10 milliTorr = 1.3 Pa.
***The end of the freeze drying cycle is tested by determining a pressure rise test value in the freeze dryer. The value should not be more than 30 milliTorr (4 Pa).

9) After completion of lyophilization, the vacuum is released by introduction of nitrogen and then vials are stoppered completely by hydraulic pressing. The vials are further sealed with flip-off seals.

10) The vials are cleaned externally and stored at 15-30° C.

The vials of Example 4 and 5 are stored at 40° C. and 75% RH for 3 months and analyzed for pH, water content (by Karl-Fisher), drug assay, and drug-related impurities. The results are tabulated below, where impurity concentrations and drug assays are percentages of the label pemetrexed content.

| | Example 4 | | Example 5 | |
|---|---|---|---|---|
| Parameter | Initial | 3 Months | Initial | 3 Months |
| pH (2.5% w/v | 7.21 | 7.02 | 7.19 | 6.95 |
| solution) | | | | |
| Water (% w/w) | 1.8 | 3.02 | 2.4 | 3.77 |
| Drug assay (by | 102.5 | 98.1 | 102.4 | 98.3 |
| HPLC) | | | | |

Impurities

| | | | | |
|---|---|---|---|---|
| Impurity 7 | 0.03 | 0.09 | 0.04 | 0.09 |
| Impurity 8 | ND | ND | ND | ND |
| Highest unidentified | 0.15 | 0.11 | 0.16 | 0.11 |
| impurity | | | | |
| Total impurities | 0.48 | 0.73 | 0.54 | 0.71 |

ND = not detected.

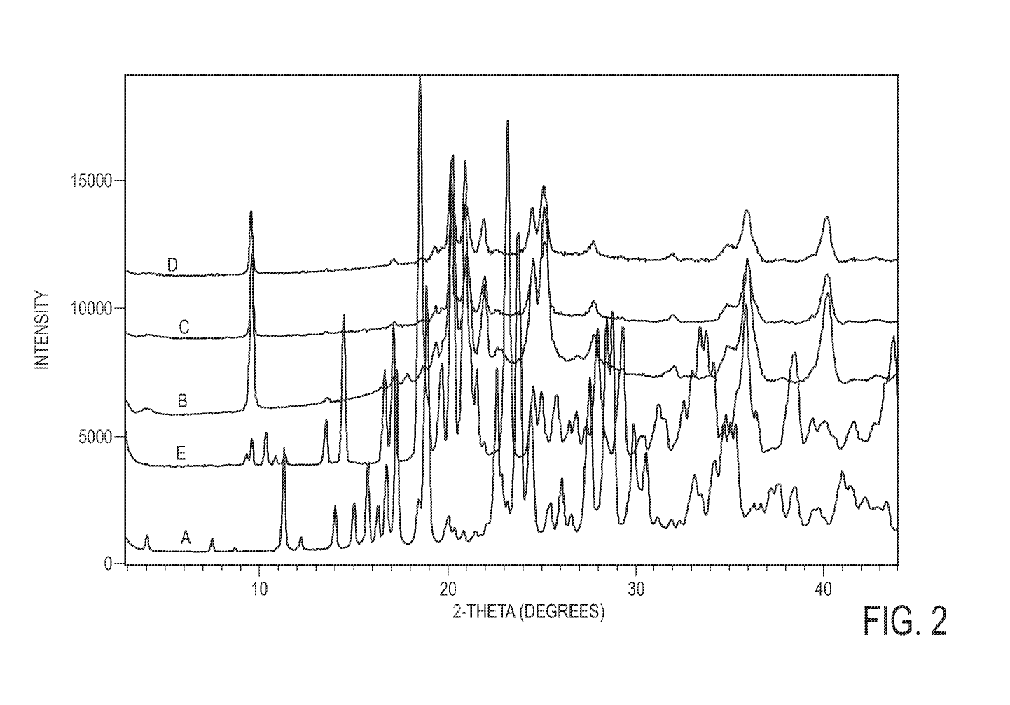

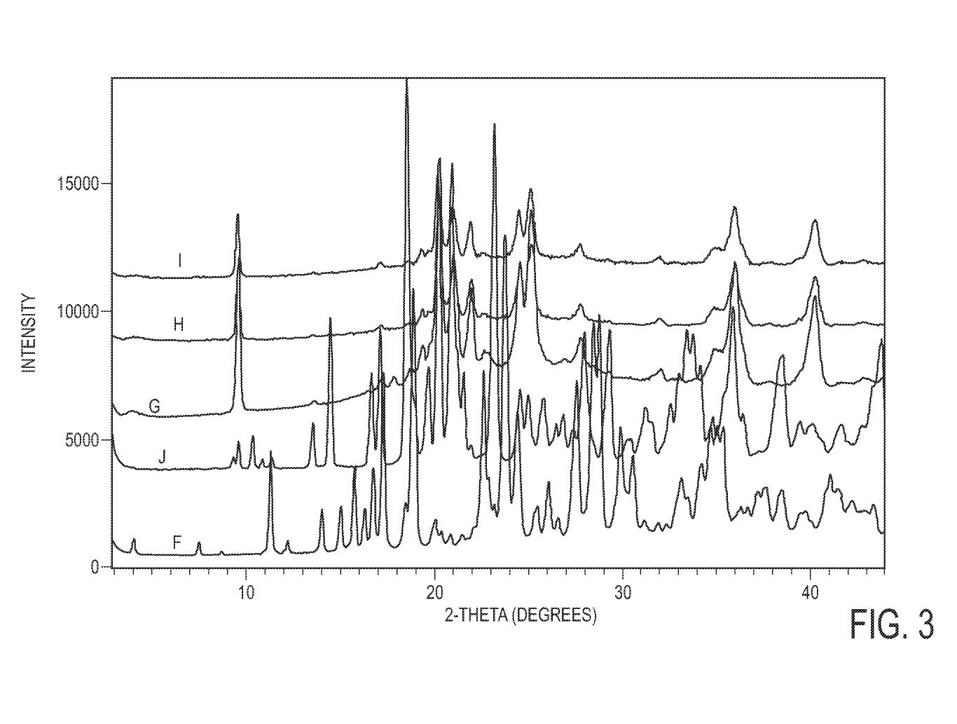

Samples from Examples 4 and 5 are stored at 40° C. and 75% RH, and at 30° C. and 75% RH, for 3 months and then analyzed by XRD. Comparative XRD patterns of the formulations from Example 4 and 5 as prepared, the formulations after three months of storage, the starting pemetrexed disodium, and similarly prepared formulations that omit pemetrexed disodium, are shown in FIGS. 2 and 3. These figures show that the polymorphic form of the starting crystalline pemetrexed disodium is not present in the formulations. In other words, pemetrexed disodium is substantially amorphous in the formulations as prepared, and remains amorphous during the storage.

1. A solid pharmaceutical formulation comprising amorphous pemetrexed, or a salt thereof, and at least one pharmaceutically acceptable excipient.

2. The solid pharmaceutical formulation according to claim 1 wherein pemetrexed or a salt thereof comprises pemetrexed disodium.

3. The solid pharmaceutical formulation according to claim 1, wherein pemetrexed or a salt thereof is at least 50 percent amorphous.

4. The solid pharmaceutical formulation according to claim 1, having a moisture content less than about 8 percent by weight.

5. The solid pharmaceutical formulation according to claim 1, having a moisture content less than about 6 percent by weight.

6. The pharmaceutical formulation according to claim 1, wherein pH of a 2.5 percent w/v solution in water is about 5 to about 8.

7. The solid pharmaceutical formulation according to claim 1, being substantially free of a sugar.

8. The solid pharmaceutical formulation of claim 1, being a lyophilized mixture comprising pemetrexed disodium and a pharmaceutically acceptable carrier.

9. The solid pharmaceutical formulation of claim 1, being a lyophilized mixture comprising pemetrexed disodium and a sugar alcohol.

10. The solid pharmaceutical formulation of claim 1, being a lyophilized mixture comprising pemetrexed disodium and mannitol.

11. The solid pharmaceutical formulation of claim 1, being a lyophilized mixture comprising pemetrexed disodium, a sugar alcohol, and a pH buffer.

12. A pharmaceutical formulation comprising pemetrexed or a salt thereof, or a hydrate, wherein an X-ray diffraction pattern of the formulation does not contain diffraction peaks that allow calculation of a ‘d’ spacing about 7.78±0.04 Å.

13. The pharmaceutical formulation of claim 1, wherein total pemetrexed-related impurities are less than about 3 percent by weight of a label pemetrexed content.

14. A process for preparing a pharmaceutical formulation comprising pemetrexed or a salt thereof, or a hydrate thereof, and at least one pharmaceutically acceptable excipient, comprising removing solvent from a solution comprising pemetrexed or a salt thereof to produce a product having pemetrexed or a salt thereof substantially in amorphous form.

15. The process of claim 14, wherein a solution further comprises a pharmaceutically acceptable carrier.

16. The process of claim 14, wherein a solution further comprises a sugar alcohol.

17. The process of claim 14, wherein a solution further comprises mannitol.

18. The process of claim 14, wherein a solution further comprises a sugar alcohol and a pH buffer.

19. The process of claim 14, wherein a solution comprises water.

20. The process according to claim 14, comprising lyophilization.

21. The pharmaceutical formulation of claim 12, wherein total pemetrexed-related impurities are less than about 3 percent by weight of a label pemetrexed content.
